(19) United States
(12) Patent Application Publication
(10) Pub. No.: US 2020/0276038 A1
(43) Pub. Date: Sep. 3, 2020

(54) APPARATUS AND METHODS FOR RESTORING TISSUE

(71) Applicant: Alucent Biomedical, Inc., Salt Lake City, UT (US)

(72) Inventors: D. H. PERKINS, Salt Lake City, UT (US); RB Eugene HAYES, Salt Lake City, UT (US); Robert R. SCOTT, Salt Lake City, UT (US)

(73) Assignee: Alucent Biomedical, Inc.

(21) Appl. No.: 16/290,363

(22) Filed: Mar. 1, 2019

(51) Int. Cl. A61F 2/958; A61L 27/50

(52) U.S. Cl. CPC A61F 2/958; A61F 2/954; A61L 27/507

(57) ABSTRACT

An apparatus and methods tissue restoration are provided. The apparatus may include a catheter shaft extending from a proximal end to a distal tip and a translucent first distal balloon positioned on a translucent distal segment of the catheter shaft proximal to the distal tip in fluid communication with a drug source via a first lumen, the first distal balloon may include first and second outer surfaces, and a patterned outer profile of first distal balloon formed by the first outer surface and the second outer surface. A second distal balloon positioned inside of and concentric with the first distal balloon. A first light fiber and a second light fiber each positioned in the catheter shaft and extending through the translucent distal segment. The drug source provides at least one drug to the first distal balloon via the first lumen.

### BACKGROUND

### Technical Field

The present disclosure generally relates to apparatus and methods to restore a tissue's function. More particularly, and without limitation, the disclosed embodiments relate to catheters, and catheter systems to create a natural vessel scaffolding.

### Background Description

Balloon catheters are used in a number of surgical applications including occluding blood flow either distally or proximally of a treatment site. The inflation of the balloon must be controlled in order to avoid over expansion or rupture of the balloon, which may rupture or otherwise damage the vessel. Percutaneous Transluminal Angioplasty (PTA), in which a balloon is used to open obstructed arteries, has been widely used to treat atherosclerotic lesions. However, this technique is limited by the vexing problems of re-occlusion and restenosis. Restenosis results from the excessive proliferation of smooth muscle cell (SMC), and the rate of restenosis is above 20%. Thus, about one in five patients treated with PTA must be treated again within several months.

Additionally, stenting is a popular treatment, in which an affected area of the artery having been constricted as a result of progress of arteriosclerosis is mechanically expanded with the aid of a balloon catheter, followed by placement of a metallic stent within the vascular lumen to restore the flow of blood. Constriction or occlusion of the artery is problematic and can be itself, or cause, major health complications. Placement of a metallic stent has been found to result in 20% to 30% of patients requiring postoperative treatment. One cause of this high frequency of required postoperative treatment is vascular intimal hyperplasia within the vascular lumen resulting in lumen narrowing despite the stent being placed. In order to decrease in-stent restenosis, attempts have been made to design a stent of a type having a surface carrying a restenosis-inhibiting drug so that when the stent is placed in an artery, the drug is eluted in a controlled manner within the vascular lumen. Those attempts have led to commercialization of drug-eluting stents (hereinafter referred to as DES) utilizing sirolimus (immunosuppressor) and paclitaxel (cytotoxic antineoplastic drug). However, since those drugs have an effect of inhibiting the proliferation of vascular cells (endothelial cells and smooth muscle cells) by acting on the cell cycle thereof, not only can the vascular intimal hyperplasia resulting from an excessive proliferation of the smooth muscle cells be suppressed, but proliferation is also suppressed of endothelial cells once denuded during placement of the stent, resulting in the adverse effect that the repair or treatment of the intima of a blood vessel becomes reduced. In view of the fact that thrombosis tends to occur more easily at a site less covered with endothelial cells in the intima of a blood vessel, an antithrombotic drug must be administrated for a prolonged time, say, half a year or so and, even though the antithrombotic drug is administrated, the drug will run out and leading to a risk of late thrombosis and restenosis.

The technical problem underlying the present disclosure is therefore to overcome these prior art difficulties by creating devices providing for controlled delivery and aspiration of therapeutic agents to the surrounding tissues, casting the tissue to a final shape, and activating the therapeutic agent in the tissue forming the cast shape and propping the vessel open. The solution to this technical problem is provided by the embodiments characterized in the claims.

### SUMMARY

The embodiments of the present disclosure include catheters, catheter systems, and methods of forming a tissue scaffolding using catheter systems. Advantageously, the exemplary embodiments allow for controlled, uniform delivery of therapeutic agents to the surrounding tissues, casting the tissue to a final shape, and activating the therapeutic agent in the tissue forming the cast shape and propping the vessel open. The tissue may be a vessel wall of a vessel within the cardiovascular system.

An apparatus is provided according to an embodiment of the present disclosure. The apparatus may include a catheter shaft extending from a proximal end to a distal tip, a first distal balloon positioned on a translucent distal segment of the catheter shaft proximal to the distal tip, the first distal balloon in fluid communication with a drug source via a first lumen. The first distal balloon may include: a translucent material, a first outer surface positioned at a first radial distance from a center of the first distal balloon, a second outer surface positioned at a second radial distance from the center of the first distal balloon, the second radial distance being larger than the first radial distance, and a patterned outer profile of first distal balloon formed by the first outer surface and the second outer surface. The apparatus may further include a second distal balloon positioned inside of and concentric with the first distal balloon, the second distal balloon in fluid communication with a second lumen separate from the first lumen. The apparatus may further include a first light fiber and a second light fiber each positioned in the catheter shaft and extending through the translucent distal segment. The drug source may provide at least one drug to the first distal balloon via the first lumen.

In some embodiments, the patterned outer profile formed by the second outer surface includes longitudinal zones favoring less resistance than other areas of the first distal balloon, the longitudinal zones permitting the first distal balloon to selectively fold along these zones as the first distal balloon may be compressed into a smaller shape. The patterned outer profile may include longitudinal and circumferential surfaces that, with the first distal balloon expanded, engage and separate plaque along a wall of a vessel of a subject into smaller, less pressure resistant and isolated sections.

In some embodiments, the longitudinal and circumferential surfaces may be interconnected and define at least one confined volume on the outer surface of the first distal balloon, the confined volume defined by a difference in radial distance from the center of the first distal balloon between the first outer surface and the second outer surface. Accordingly, expanding the first distal balloon causes the confined volume to be sealed against the vessel wall, minimizing drug loss to smaller vessels, side branches or collaterals. Expanding the first distal balloon may create isolated plaque sections allowing the drug to penetrate a vessel wall through gaps between each of the isolated plaque sections.

In some embodiments, the first outer surface includes slitted apertures that communicate the drug from the first distal balloon to a treatment area of a subject. In some embodiments, the second outer surface includes slitted apertures that communicate the drug from the first distal balloon to a treatment area of a subject.

In some embodiments, the first distal balloon may comprise a confined volume in fluid communication with the proximal end of the catheter shaft. The confined volume may be in fluid communication with at least one slitted aperture extending through from an interior side of the first distal balloon to an exterior side of the first distal balloon providing fluid communication from the interior side of the first distal balloon to the exterior side of the first distal balloon. The at least one slitted aperture may be in fluid communication with the drug source, the drug source supplying a drug into the confined volume in the first distal balloon and through the slitted aperture to the exterior side of the first distal balloon.

In some embodiments, a volume pressure of the first distal balloon increases and inflates the first distal balloon, the increased volume pressure forces edges of the slitted aperture to open apart thereby reducing the balloon pressure. In some embodiments, the second outer surface of the first distal balloon contains a drug secured to the surface permitting the simultaneous delivery of two distinct and separate drugs.

In some embodiments, expanding the second distal balloon consequently expands the first distal balloon as an outer surface of the second distal balloon contacts an inner surface of the first distal balloon. The translucent material of the distal segment, the first distal balloon, and the second distal balloon is transparent. The first light fiber and the second light fiber provide light activation through the distal segment, the first distal balloon, and the second distal balloon.

According to another embodiment of the present disclosure a method of tissue restoration in a blood vessel of a subject is provided. The method may include providing a catheter into the blood vessel, the catheter may include the features of the apparatus described herein. The catheter may include a catheter shaft extending from a proximal end to a distal tip, a first distal balloon positioned on a translucent distal segment of the catheter shaft proximal to the distal tip, the first distal balloon in fluid communication with a drug source via a first lumen, the first distal balloon including: a translucent material, a first outer surface positioned at a first radial distance from a center of the first distal balloon, a second outer surface positioned at a second radial distance from the center of the first distal balloon, the second radial distance being larger than the first radial distance; a patterned outer profile of first distal balloon formed by the first outer surface and the second outer surface. The catheter may include a second distal balloon positioned inside of and concentric with the first distal balloon, the second distal balloon in fluid communication with a second lumen separate from the first lumen; and a first light fiber and a second light fiber each positioned in the catheter shaft and extending through the translucent distal segment. The method may further include supplying a drug from the drug source to the first distal balloon; partially expanding the second distal balloon; expanding the first distal balloon into contact with the blood vessel in a treatment area; delivering the drug to the treatment area through at least one slitted aperture in the first distal balloon; fully expanding the second distal balloon; activating the first light fiber and the second light fiber thereby providing light transmission through the distal segment, the first distal balloon, and the second distal balloon to activate the drug in the treatment area.

In some embodiments, the method may include engaging and separating plaque along a wall of a vessel of a subject into smaller, less pressure resistant and isolated sections using the longitudinal and circumferential surfaces of the patterned outer profile. The longitudinal and circumferential surfaces may be interconnected and define at least one confined volume on the outer surface of the first distal balloon, the confined volume defined by a difference in radial distance from the center of the first distal balloon between the first outer surface and the second outer surface. The method may include creating isolated plaque sections that allow the drug to penetrate a vessel wall through gaps between each of the isolated plaque sections. The method may include expanding the second distal balloon thereby expanding the first distal balloon as an outer surface of the second distal balloon contacts an inner surface of the first distal balloon.

According to another embodiment, an apparatus is provided. The apparatus may include a catheter shaft extending from a proximal end to a distal tip, a first distal balloon positioned on a translucent distal segment of the catheter shaft proximal to the distal tip, the first distal balloon in fluid communication with a drug source via a first lumen The first distal balloon may include: a translucent material; a first outer surface positioned at a first radial distance from a center of the first distal balloon; a second outer surface positioned at a second radial distance from the center of the first distal balloon, the second radial distance being larger than the first radial distance; a patterned outer profile of first distal balloon formed by the first outer surface and the second outer surface, the patterned outer profile including interconnected longitudinal and circumferential surfaces that define at least one confined volume on the outer surface of the first distal balloon, the confined volume defined by a difference in radial distance from the center of the first distal balloon between the first outer surface and the second outer surface. The apparatus may include a first light fiber and a second light fiber each positioned in the catheter shaft and extending through the translucent distal segment; wherein the drug source provides at least one drug to the first distal balloon via the first lumen the interconnected longitudinal and circumferential surfaces, with the first distal balloon expanded, engage and separate plaque along a wall of a vessel of a subject into smaller, less pressure resistant and isolated sections.

Additional features and advantages of the disclosed embodiments will be set forth in part in the description that follows, and in part will be obvious from the description, or may be learned by practice of the disclosed embodiments. The features and advantages of the disclosed embodiments will be realized and attained by the elements and combinations particularly pointed out in the appended claims.

It is to be understood that both the foregoing general description and the following detailed description are examples and explanatory only and are not restrictive of the disclosed embodiments as claimed.

The accompanying drawings constitute a part of this specification. The drawings illustrate several embodiments of the present disclosure and, together with the description, serve to explain the principles of the disclosed embodiments as set forth in the accompanying claims.

### BRIEF DESCRIPTION OF THE DRAWINGS

FIG. 1 is a side elevational view of an exemplary catheter, according to embodiments of the present disclosure.

FIG. 2A is a side elevational view of a distal portion of the catheter of FIG. 1.

FIG. 2B is a perspective partial section view of the exemplary catheter of FIG. 2A.

FIG. 3 is a perspective view of an exemplary catheter, according to another exemplary embodiment of the present disclosure.

FIG. 4A is a cross-sectional view taken along line 4A-4A of FIG. 2A.

FIGS. 4B and 4C are cross-sectional views taken along line 4B-4B of FIG. 2A, removing portions of the internal structure.

FIG. 5 is another view of the cross-sectional view taken along line 4A-4A of FIG. 2A, removing portions of the internal structure

FIG. 6 is a perspective view of the cross-sectional view of FIG. 5, removing portions of the internal structure.

FIG. 7 shows a series of cross-sectional views showing an exemplary catheter according to embodiments of the present disclosure.

FIG. 8 is a schematic plan view of a patterned outer surface according to an exemplary embodiment of the present disclosure.

FIG. 9 is a perspective view of a cross-sectional view removing portions of the internal structure, according to an exemplary embodiment of the present disclosure.

FIGS. 10A to 10E are perspective views of exemplary catheters according to embodiments of the present disclosure.

FIG. 11 is a perspective view of a cross-sectional view removing portions of the internal structure, according to an exemplary embodiment of the present disclosure.

FIGS. 12A to 12E are perspective views of exemplary catheters according to embodiments of the present disclosure.

FIG. 13 is a side elevational view of an exemplary catheter placed in a vessel of a subject, according to an exemplary embodiment of the present disclosure.

FIG. 14 is a side elevational view of an exemplary apparatus placed in a vessel of a subject, according to another exemplary embodiment of the present disclosure.

### DETAILED DESCRIPTION

Reference will now be made in detail to embodiments and aspects of the present disclosure, examples of which are illustrated in the accompanying drawings. Where possible, the same reference numbers will be used throughout the drawings to refer to the same or like parts.

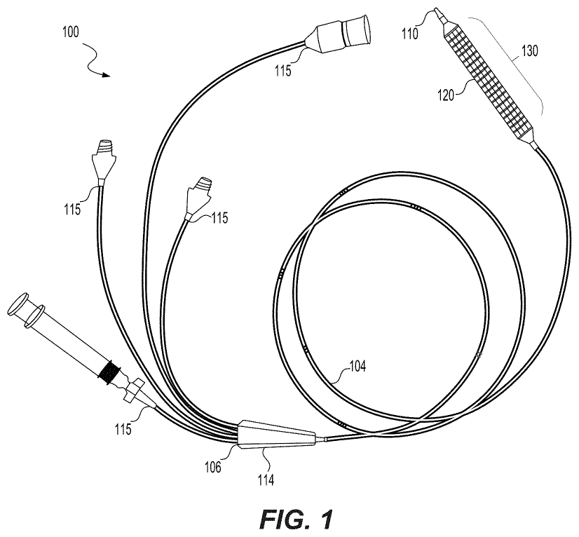

FIG. 1 illustrates an apparatus 100 in accordance with an embodiment of this disclosure. The apparatus 100 having a catheter shaft 104 that extends from a proximal end 106 to a distal tip 110 of the apparatus 100. The apparatus 100 may be configured for longitudinal movement and positioning within a vessel (e.g. blood vessel) of a subject. In some embodiments, the apparatus 100 may be configured for occlusion of the vessel and treatment of an area of the vessel. For example, the apparatus 100 may be configured for occlusion of a blood vessel and delivery of a drug to the occluded area of the vessel and forming and casting a shape in the vessel, as will be described in more detail below.

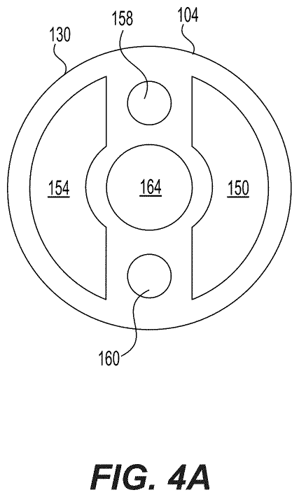

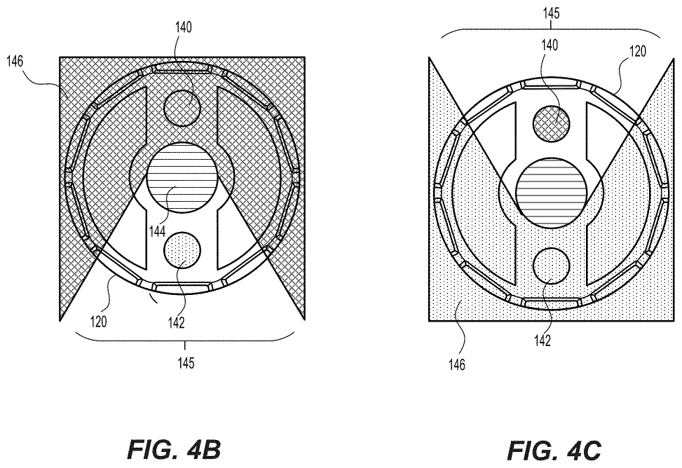

The apparatus 100 may include a proximal end connector 114 positioned at the proximal end of the apparatus 100, and the catheter shaft 104 may extend in a distal direction therefrom. The catheter shaft 104 may define a plurality of lumens that are accessible via a plurality of ports the proximal end connector 114. The plurality of ports 115 may be configured to engage with external sources desirable to communicate with the plurality of lumens. The ports may engage with external sources via a variety of connection mechanisms, including, but not limited to, syringes, over-molding, quick-disconnect connectors, latched connections, barbed connections, keyed connections, threaded connections, or any other suitable mechanism for connecting one of the plurality of ports to an external source. Non-limiting examples of external sources may include inflation sources (e.g. saline solutions), gaseous sources, treatment sources (e.g. medication, drugs, or any desirable treatment agents discussed further below), light sources, among others. In some embodiments, apparatus 100 can be used with a guide wire (not shown), via guide wire lumen 164 (see FIG. 4A), to assist in guiding the catheter shaft 104 to the target area of the vessel.

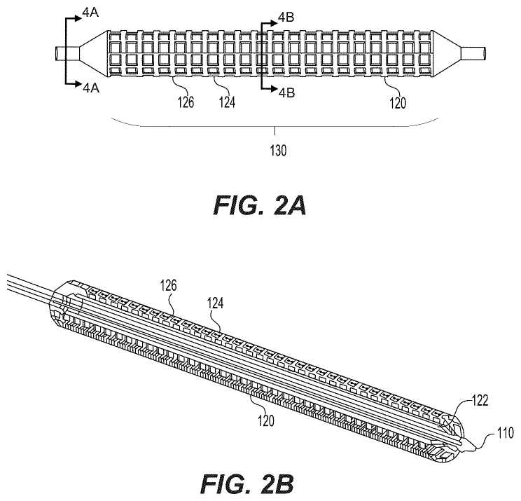

FIGS. 1, 2A, and 2B illustrate the apparatus 100 may include a first distal balloon 120 positioned over a distal segment 130 of the catheter shaft 104 proximal to the distal tip 110. In some embodiments, the first distal balloon 120 may be proximally offset from the distal tip 110 a distance between 0 mm and 1 mm, 0 mm and 2 mm, 0 mm and 3 mm, 0 mm and 10 mm, or 0 and 50 mm. In some embodiments, the first distal balloon 120 may inflate to 2 to 10 millimeters (mm) in diameter. In other embodiments, the first distal balloon 120 may inflate to 2 to 4 cm in diameter. The first distal balloon 120 may have a length of about 0.5 to 1 centimeters (cm), 1 to 2 cm, 1 to 3 cm, or 1 to 5 cm, or 1 to 10 cm, or 1 to 15 cm, or 1 to 20 cm, or 1 to 25 cm, and may take any shape suitable for supporting a wall of a blood vessel of the subject when the non-compliant or semi-compliant balloon is inflated. For example, the first distal balloon 120 may expand into a cylindrical shape surrounding the distal segment 130 of the catheter shaft 104. The cylindrical shape may be gradually tapered inward at a proximal end and a distal end of the first distal balloon 120, thereby providing a gradually tapered proximal end and distal end of the first distal balloon 120 that taper into contact with and become flush with the catheter shaft 104. The first distal balloon 120 may include a first outer surface 124 and a second outer surface 126. The first outer surface 124 is positioned a radial distance from the center of the first distal balloon 120. The second outer surface 126 is positioned more radially distant from the first outer surface 124. The radii of both surfaces 124, 126 may vary, but remain equidistant from one another, forming a nonuniform spheroidal shape of the first distal balloon 120.

Non-limiting examples of shapes the inflated first distal balloon 120 may form include a cylindrical shape, football-shaped, spherical, ellipsoidal, or may be selectively deformable in symmetric or asymmetric shapes so as to limit the potential difference in the treated vessel shape and the untreated vessel shape reducing edge effects common between two surfaces of different stiffness as found in metal stents. The force exerted against a vessel interior by first distal balloon 120 may be strong enough to scaffold the vessel wall with the apparatus 100 held in a stationary position within the vessel or other hollow body structure. However, the force is not so great as to damage the interior surface of the vessel or other hollow body structure. The first distal balloon 120 may be substantially translucent

The apparatus 100 may further include a second distal balloon 122 positioned inside of and concentric with the first distal balloon 120. The second distal balloon 122 may have one continuous surface sealed at each end around the catheter shaft 104 forming an enclosed volume and in fluid communication through a port with the catheter shaft 104 through a distinct and separate lumen from the first distal balloon 122 as will be described in more detail below. The second distal balloon 122 may be substantially translucent. The second distal balloon 122 may be positioned concentric with and inside of the first distal balloon 120 such that inflating the second distal balloon 122 may also expand the first distal balloon 120 as an outside surface of the second distal balloon 122 contacts an inside surface of the first distal balloon 120.

The apparatus 100 may include a plurality of connectors 115 positioned proximally to the proximal end connector 114. For example, the first distal balloon 120 may be terminated at the proximal end with a connector capable of receiving a drug source. In some embodiments, the connector may be a luer configuration. The second distal balloon 122 may be terminated at the proximal end with a separate and distinct connector capable of receiving a fluid for inflation, which may, in some embodiments, be a luer configuration. A center lumen (discussed in more detail below), may be terminated at the proximal end with a connector capable of receiving a fluid source for clearing the lumen from the proximal termination to outside the distal tip, and in some embodiments may include a luer configuration. The center lumen may also accommodate a guidewire for tracking the catheter apparatus to the desired anatomical location. As discussed in more detail below, the apparatus 100 may also include light fibers that may be terminated at the proximal end with an adaptor capable of connecting with a light source. Each light fiber may terminate with a separate and distinct adaptor or both light fibers may share an adaptor to a light source.

The materials of the apparatus 100 may be biocompatible. The catheter shaft 104 may include material that is extrudable and capable of sustaining lumen integrity. The distal segment 130 of the catheter shaft 104 is substantially translucent to allow light transmission from light fibers. The catheter shaft 104 material is rigid enough to track over a guidewire and soft enough to be atraumatic. The catheter shaft 104 may be made of materials including, but not limited to polymers, natural or synthetic rubber, metal and plastic or combinations thereof, nylon, polyether block amide (PEBA), nylon/PEBA blend, thermoplastic copolyester (TPC), a non-limiting example may be Hytrel®, and polyethylene. The shaft materials can be selected so as to maximize column strength to the longitudinal length of the shaft. Further, the shaft materials can be braided, so as to provide sufficient column strength. The shaft materials can also be selected so as to allow the device to move smoothly along a guide wire. The catheter shaft 104 can also be provided with a lubricious coating as well as antimicrobial and antithrombogenic coatings. The shaft materials should be selected so as not to interfere with the efficacy of the agent to be delivered or collected. This interference may take the form of absorbing the agent, adhering to the agent or altering the agent in any way. The catheter shaft 104 of the present disclosure may be between about 2-16 French units (“Fr.” where one French equals ⅓ of a millimeter, or about 0.013 inches). The catheter shafts to be used in coronary arteries may be between about 3-5 Fr. in diameter, and more specifically may be 3 Fr. The catheter shafts to be used in peripheral vessels may be between about 5-8 Fr. in diameter, and more specifically 5 Fr. The catheter shafts to be used in the aorta may be between about 8-16 Fr. in diameter, and more specifically 12 Fr.

The first distal balloon 120 and the second distal balloon 122 may be substantially translucent permitting light from light fibers to be transmitted substantially beyond the inflated diameters of the first distal balloon 120. The compliance of the first distal balloon 120 and the first outer surface 124 and second surface 126 and the second distal balloon 122 may be comparable or dissimilar. For example, the first distal balloon 120 may be compliant such that the material conforms substantially to a vessel's morphology. The second distal balloon 122 material may be more rigid and noncompliant capable of higher internal pressures with minimal outward expansion for opening vessels more resistant to pressures.

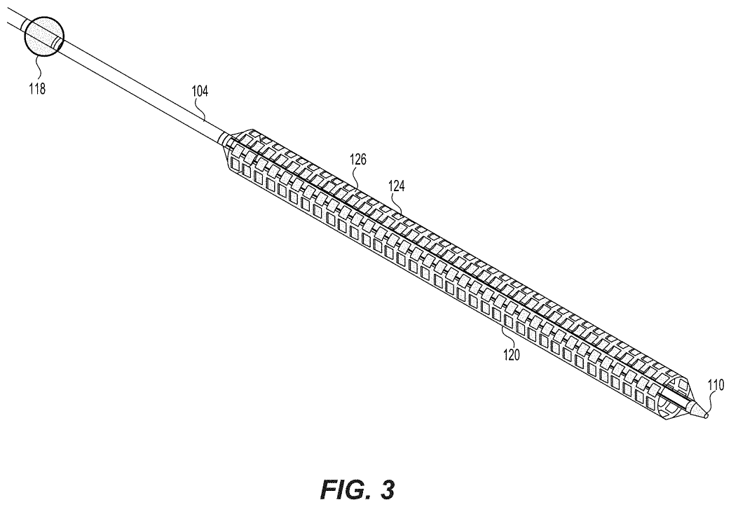

FIG. 3 is a perspective view of another embodiment of the present disclosure that may include a proximal balloon 118 in fluid communication with an additional lumen of the catheter shaft 104. The proximal balloon 118 may be positioned on the catheter shaft 104 proximal to the first distal balloon 120. In some embodiments, proximal balloon 118 may inflate to 2 to 10 millimeters (mm) in diameter. In other embodiments, the proximal balloon 118 may inflate to 3 to 5 centimeters (cm) in diameter. The proximal balloon 118 may have a length of about 1 to 2 centimeters (cm) and may take any shape suitable for occluding and sealing a blood vessel of the subject when a compliant or semi-compliant balloon is inflated. Non-limiting examples of shapes the inflated balloon may form include oblong, football-shaped, spherical, ellipsoidal, or may be selectively deformable in symmetric or asymmetric shapes. The force exerted against a vessel interior by the proximal balloon 118 may be strong enough to hold the catheter assembly 100 in a stationary position within the vessel or other hollow body structure and provide an adequate seal to control blood or fluid flow. However, the force is not so great as to damage the interior surface of the vessel or other hollow body structure.

FIG. 4A is a cross-sectional view taken along line 4A-4A of FIG. 2A showing a plurality of lumens within the assembly 100, according to an embodiment of this disclosure. The catheter shaft 104 may have an outside diameter and outside surface 130. The catheter shaft 104 may have an inside configuration of five distinct and separate lumens, extending from the proximal end 106 to the distal tip 110.

The first distal balloon 120 may be in fluid communication with a first distal balloon inflation lumen 150. The second distal balloon 122 may be in fluid communication with a second distal balloon inflation lumen 154 that is separate and distinct from the first distal balloon inflation lumen 150. The first distal balloon 120 may be in fluid communication with an inflation source via the first distal balloon inflation lumen 150 separate from the second distal balloon inflation lumen 154. The first distal balloon inflation lumen 150 may extend through the catheter shaft 104 and have an input at one of the plurality of ports 115 of the proximal end connector 114. Fluid communication between the first distal balloon 120 and the inflation source via the first distal balloon inflation lumen 150 may cause the first distal balloon 120 to selectively inflate and deflate separately from and independently of the second distal balloon 122. Similarly, the second distal balloon 122 may be in fluid communication with an inflation source via the second distal balloon inflation lumen 154 separate from the first distal balloon inflation lumen 150. Fluid communication between the second distal balloon 122 and the inflation source via the second distal balloon inflation lumen 154 may cause the second distal balloon 122 to selectively inflate and deflate separately from and independently of the first distal balloon 120.

A first light fiber lumen 158 and a second light fiber lumen 160 may be positioned in the catheter shaft 104 to receive light fibers, and the first light fiber lumen 158 and the second light fiber lumen 160 may extend from the proximal end 106 into the distal segment 130, and may be positioned substantially symmetric, longitudinally opposed and parallel one to another within the catheter shaft 104. In another exemplary embodiment, the catheter shaft 104 may include a single light fiber lumen. In still other embodiments, the catheter shaft 104 may include a plurality of light fiber lumens.

A guidewire lumen 164 may be concentric with the catheter shaft outside diameter and may be arranged in the catheter shaft 104, from the proximal end 106 through the distal tip 110. The guidewire lumen 164 may accommodate a guidewire to aid the placement of the apparatus 100 to a desired anatomical position communicating with the proximal end and distal tip. The guidewire may be separate and distinct from the apparatus 100 and extend proximally beyond the proximal end and distally beyond the distal tip of the catheter shaft. The guidewire lumen 164 is located concentric with the catheter outer diameter; the catheter shaft is oriented concentrically with the guidewire permitting the catheter shaft 104 to follow the guidewire without favoring one side of the catheter shaft 104 or whipping from side to side. The guidewire may remain in the guidewire lumen 104 maintaining anatomical position during the activation of the light fibers.

FIGS. 4B and 4C illustrate cross-sectional views taken along line 4B-4B of FIG. 2A. The apparatus 100 may further include a first light fiber 140 and a second light fiber 142 positioned in the catheter shaft 104 and extending through the distal segment 130. The light fibers 140, 142 may transmit light through the distal segment 130, the second distal balloon 122, the first distal balloon 120. The light fiber 140 may be connected to the proximal end connector 114 and may have proximal ends that connect to a light fiber activation source via at least one of the plurality of ports 115. In some embodiments, the light fibers 140, 142 may be configured to transmit light at a wavelength of 375 nanometers (nm) to 475 nm, and more specifically 450 nm that transmits through the distal segment 130 and the first distal balloon 120. In some embodiments, the light first fiber 140 may be positioned in the first light fiber lumen 158 and the second light fiber 142 may be positioned in the second light fiber lumen 160.

In some embodiments, light from the light fibers 140, 142 may be unable to penetrate through a guidewire 144 forming a shadow 145 opposite the light and beyond the guidewire 144. Accordingly, the light fibers 140, 142 may each generate a respective light transmission area 146. The light fiber lumens 158, 160 are oriented substantially opposite one another minimizing the shadow 145 formed by the light impenetrable guidewire 144, permitting the transmission of light to penetrate the circumference of the catheter shaft 104 from the first light fiber 140 or the second light fiber 142. In another embodiment, the catheter shaft 140 may include a single light fiber, and the guidewire may be removed for light penetration to the outer tissue.

In some embodiments, the light fibers 140, 142 may be made from plastic core and cladding. The refractive index of the core is high. The refractive index of the cladding is low. A non-limiting example of the core material may be polymethyl methacrylate (PMMA). A non-limiting example of the cladding may be a silicone material.

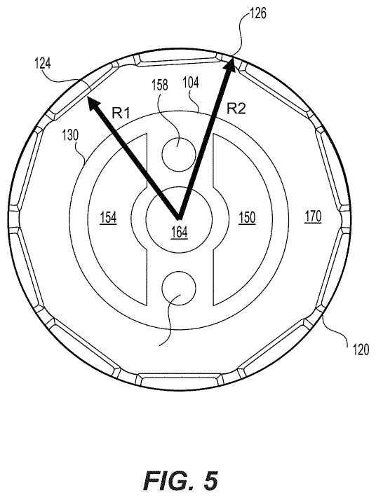

FIG. 5 is another view of the cross-sectional view taken along line 4A-4A of FIG. 2A. As discussed above, the first distal balloon 120 may have two distinct surfaces. The first outer surface 124 may be positioned a radial distance (R1) from the center of the first distal balloon 120. The second outer surface 126 may be positioned more radially distant from the first outer surface 124. The radii of both surfaces R1, R2 may vary, but remain equidistant from one another, forming a nonuniform spheroidal shape. The two surfaces 124, 126 may be two distinct components or materials.

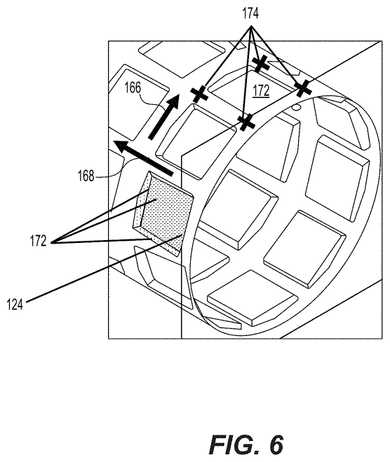

As illustrated in FIG. 6, the second outer surface 126 may form a pattern representing circumferential directed sections 166 and longitudinal directed sections 168 that may be interconnected, minimally contacting and sealing against the first outer surface 124. The interconnected circumferential sections 166 and the longitudinal sections 168 of the second outer surface pattern generate a confined volume 172 or well. The confined volume 172 is formed from the wall connecting the intersection points 174 and the sealing contact with the first outer surface 124. The pattern generated by the second outer surface 126 may not produce wells the same size or wells positioned symmetrically around the outer surface but may produce wells of a size and position suited to a function. In another embodiment, the first distal balloon 120 may contain only one distinct surface, forming no surface variation.

The pattern generated by the second outer surface 126 may have longitudinal zones (e.g. longitudinal sections 168) favoring less resistance than other areas of the first distal balloon 120, permitting the first distal balloon 120 to selectively fold along these longitudinal zones as the balloon may be compressed into a smaller shape.

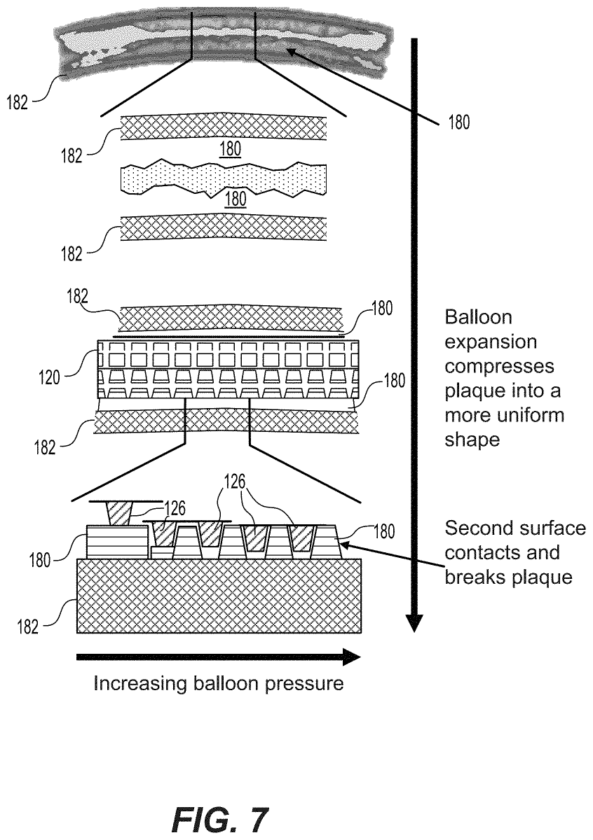

FIG. 7 shows a series of cross-sectional views illustrating an exemplary catheter according to embodiments of the present disclosure. The pattern generated by the second outer surface 126 may contain longitudinal sections 168 and circumferential sections 166 capable of engaging and separating plaque 180 covering a portion of a vessel wall 182 into smaller, less pressure resistant and isolated sections. The plaque 180 on the vessel wall 182 may be organized into a structure more rigid than the vessel wall 182, reducing the ability of the vessel wall 182 to stretch and expand circumferentially, accommodating the increased pressure of pulsatile blood flow. Inflating the first distal balloon 120, allows the pattern of the second outer surface 126 to minimally contact the plaque 180, producing a focal point force from the pressure of the inflated first distal balloon 120. The focal point force may be larger than the ultimate strength of the plaque 180 at the location of the pattern contact (e.g. the second outer surface 126), causing the plaque 180 to separate into isolated areas mirroring the shape of the isolated volume 172. The differential rigidity between the plaque 180 and the vessel wall 182, permits the separation of the plaque 180 with minimal vessel expansion. The plaque 180 may break before the vessel 182 expands. The second outer surface pattern may be selected to optimize the desired plaque shape and size. Smaller patterns may impart less vessel expansion but may break off as debris. Larger patterns may impart more vessel expansion but may remain intact with the vessel wall and impede circumferential vessel expansion. In some embodiments, the pattern shape should balance these two competing needs.

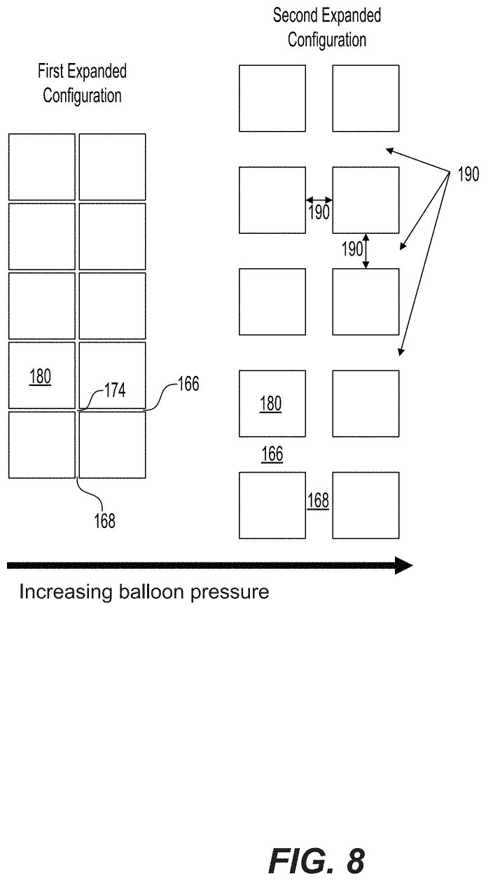

FIG. 8 is a schematic plan view of illustrating the difference between a first expanded configuration of the patterned outer surface and a second expanded configuration of the patterned outer surface. The plaque 180 covering the vessel wall 182 may impede a drug substance from penetrating the vessel wall 182. Expanding the first distal balloon 120 thereby creating isolated plaque sections, may permit a drug substance to penetrate the vessel wall 180 more easily through the gaps 190 between each of the isolated plaque sections. The first distal balloon 120 may be expanded into contact with the plaque 180 in a first expanded configuration. The first distal balloon 120 may be further expanded to a second expanded configuration. The second outer surface 126 contacts the plaque 180 and may score, break, crack, separate, or divide the plaque 180 into smaller pieces from the point forces generated at the second outer surface 126, thereby creating the gaps 190 for improved drug penetration.

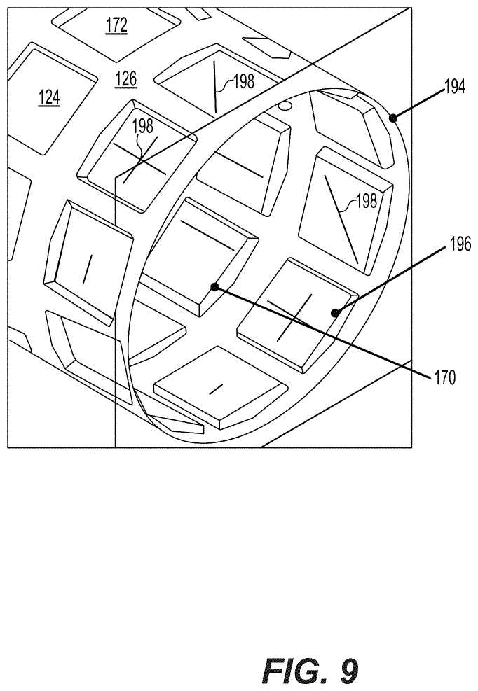

FIG. 9 illustrates the first outer surface 124 of the first distal balloon 120 may have a thickness 194 forming an outside first surface and an inside first surface 196. The inside first surface 196 forms a confined and isolated volume 170 (See also FIG. 5) in fluid communication with the proximal end 106 of the catheter shaft 104 and a plurality of slitted apertures 198. The first outer surface 124 and the second outer surface 126 are substantially translucent.

The confined volume 170 of the first distal balloon 120 may contain at least one slitted aperture 198 penetrating through from one side to the other side of the first outer surface 124. The slitted apertures 198 may be any length within the confined volume 170, but not contact the wall of the second outer surface 126. The slitted apertures 198 may be oriented in any direction of the confined volume 170 on the first outer surface 124. The slitted apertures 198 may be in fluid communication with a drug source, the drug source supplying a drug into the first distal balloon 120 volume and through the slitted aperture 198.

The scoring ability of the first distal balloon 120 with the isolated volumes 170 may permit breaking plaque for improved drug penetration. A reduction in size of the second outer surface 126 may reduce the amount of force required to separate the plaque 180, and an increased frequency of the second outer surface 126 in the patterned outer profile may cause an increased number of cracks in the plaque 180 for the drug penetration.

The edges of the slitted apertures 198 may remain together and closed as the volume 170 of the first distal balloon 120 is filled with a drug source, allowing the first distal balloon 120 to nearly fill and inflate without loss of the drug source. As the first distal balloon volume fills and inflates, the volume pressure will increase, forcing the edges of the slitted aperture 198 to open apart and fill the well 172, reducing the balloon pressure. Similarly, as the volume and corresponding pressure of the first distal balloon 120 is reduced from the filling the well 172, the edges of the slitted aperture 198 may close together and stop filling the well before the first distal balloon 120 deflates substantially preventing surrounding debris from impeding the slitted aperture 198 function. In this manner of inflating and deflating the first distal balloon 120 may control the delivery of the drug source as the wells 172 are filled and emptied.

In some embodiments, the apparatus 100 may be capable of delivery two drugs simultaneously. For example, the outside of the first distal balloon 120 may be coated with a first drug and a second drug may be delivered through the slitted apertures 198. Accordingly, the first drug and the second drug may be different drugs. In some embodiments, the first drug and the second drug may be the same drug. In a non-limiting example, the first distal balloon 120 may be coated with Paclitaxel and infusing Natural Vascular Scaffolding (NVS) through the slits and wells to the vessel wall.

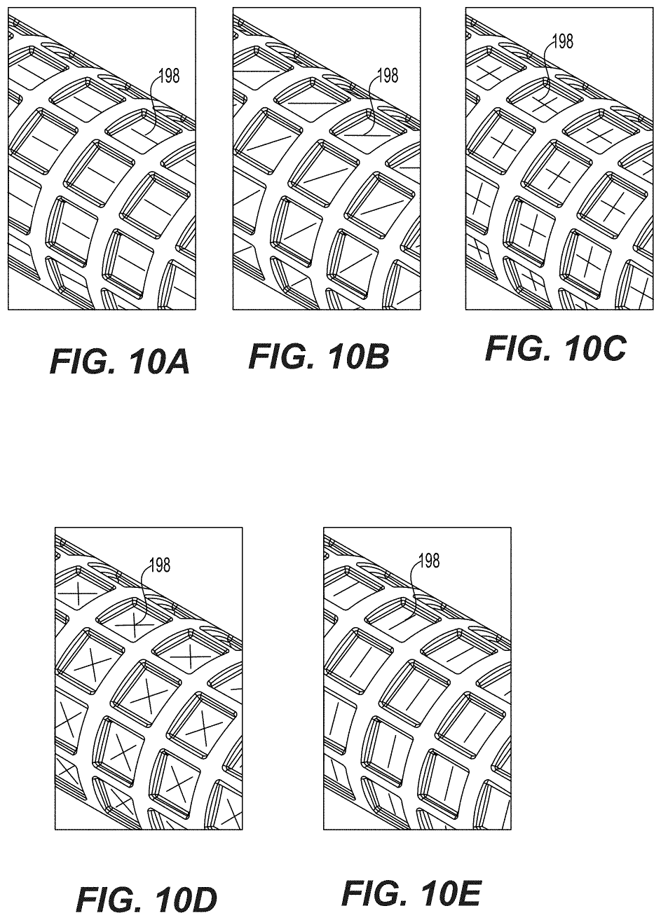

As illustrated in FIGS. 9 through 10E, the slitted apertures 198 may take a variety of shapes and orientations. For example, the slitted apertures 198 may be a single line through the well 172 which may be oriented vertically, horizontally, or at any appropriate angle therebetween. Additionally, the slitted aperture 198 may include two intersecting lines through the well 172. The lines may intersect at any appropriate angle, for example the lines may be perpendicular and meet at the mid-point of each line. The lines may not intersect or may intersect at any point along the lines. There may also be a plurality of lines arranged in any orientation.

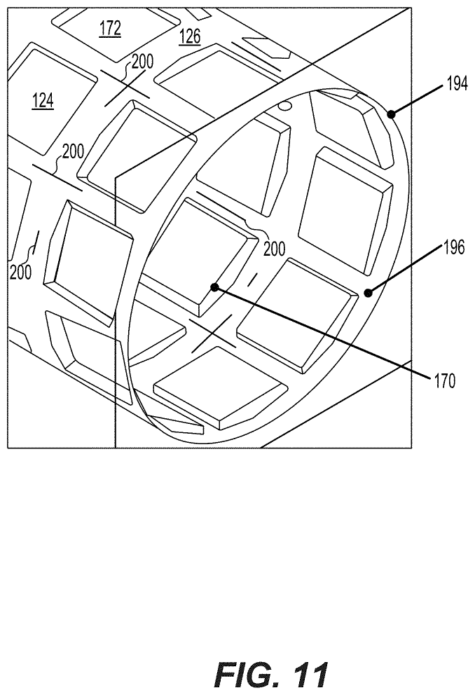

FIG. 11 illustrates the circumferential sections 166 and longitudinal sections 168 that may be interconnected sections of the second outer surface 126 of the first distal balloon 120 may contain slitted apertures 200 penetrating through from one side to the other side of the second outer surface 126, fluidly communicating from the inside volume of the first distal balloon 120 to the outside surface of the second outer surface 126. Similarly, to the slitted apertures 198 of the first outer surface 124 of the first distal balloon 120, the slitted apertures 200 on the second outer surface 126 may be any length and may be oriented in any direction. Additionally, there may be more than one slitted aperture 200 oriented within the second outer surface 126. The slitted apertures 200 may function similarly under differential pressure.

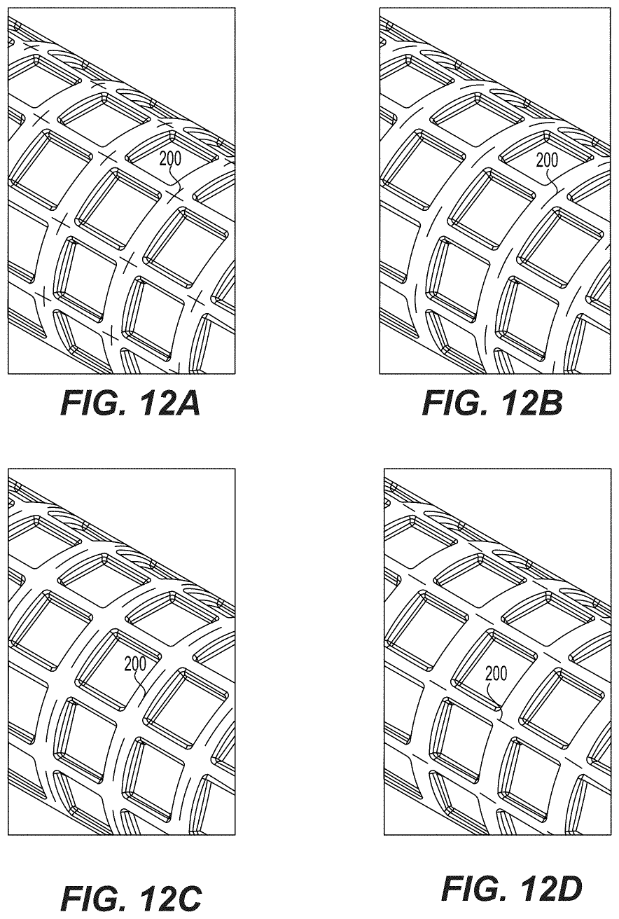

As illustrated in FIGS. 11 through 12E, the slitted apertures 200 may take a variety of shapes and orientations. For example, the slitted apertures 200 may be a single line which may be oriented vertically, horizontally, or at any appropriate angle therebetween. Additionally, the slitted aperture 200 may include two intersecting lines. The lines may intersect at any appropriate angle, for example the lines may be perpendicular and meet at the mid-point of each line. The lines may not intersect or may intersect at any point along the lines. There may also be a plurality of lines arranged in any orientation.

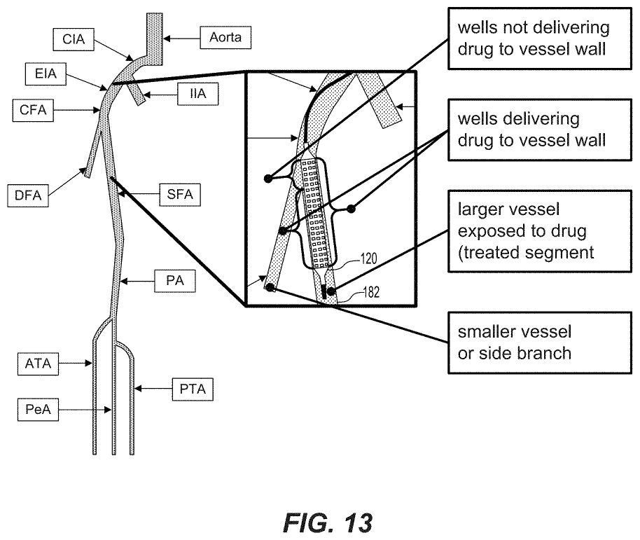

FIG. 13 is a side elevational view of an exemplary catheter placed in a vessel of a subject, according to an exemplary embodiment of the present disclosure. The target area for a delivery of drug source may be a vessel of the cardiovascular system. The first distal balloon 120 may be inflated with a drug source and expanded toward the vessel wall. The second outer surface 126 of the first distal balloon 120 may engage and seal against the vessel wall 182. The vessel wall 182 may cover and isolate the well 172, permitting the drug source to fill the well 172 when the internal pressure opens the edges of the slitted aperture (e.g. apertures 198 and/or 200), exposing the vessel wall 182 to the drug. In the event a well 172 or series of wells are positioned in the proximity of a smaller vessel, side branch or collateral, the drug from those wells may be lost to the smaller vessels. However, all the remaining wells 172 deliver drug to their adjacent vessel walls 182 such that drug is delivered uniformly to the vessel wall 182.

The second distal balloon 122 may be partially inflated before, during, or after the first distal balloon 120, reducing the volume of the first distal balloon 120. This complimentary operation of the first distal balloon 120 and second distal balloon 122, separately or simultaneously, permits reliable drug delivery to substantially different vessel anatomies. For example, the second distal balloon 122 may be inflated first, pushing the first distal balloon 120 towards the vessel wall 182. An aqueous drug may be used to inflate the first distal balloon 120 and deliver the drug while maintaining contact with the vessel wall 182 as the pressure in the first distal balloon 120 is reduced from the loss of delivered drug. Similarly, an aqueous drug may fill the volume of the first distal balloon 120 until the edges of the slitted apertures 198 open. The second distal balloon 122 may be inflated gradually maintaining the pressure in the first distal balloon 120 continuing to deliver the drug. In some embodiments, expanding the first distal balloon 120 may seal the confined volume 170 against the vessel wall 182 thereby minimizing drug loss to smaller vessels, side branches or collaterals.

The second distal balloon 122 may be inflated, subsequent to drug delivery by the first distal balloon 120, circumferentially supporting the internal surface of the vessel wall 182. While in this vessel supported position, a light source may be supplied to the light fibers 140, 142 in the catheter shaft 104 for transmittance through the catheter shaft 104, through the first distal balloon 120 and the second distal balloon 122, and into the vessel wall 182 as previously described.

There are several combinations for the local delivery of the drug source. For example, a solid drug may be coated on the second surface of the first distal balloon 120 and an aqueous drug may be delivered through the slitted apertures of the first surface of the first distal balloon. The drug may be the same, one solid and one aqueous, each penetrating the vessel wall differently. The drugs may be complimentary but different substances. The aqueous or solid drug may assist in the capacity of an excipient or activate its counterpart through a controlled reaction. Similarly, the solid drug may be coated on the first surface of the first distal balloon and an aqueous drug may be delivered through the slitted apertures on the second surface. The drugs may be dissimilar and non-complimentary affecting the vessel wall through substantially different methods of action.

Additionally, therapeutic agents useful with the device of the present disclosure include any one of or a combination of several agents which are gas, liquid, suspensions, emulsions, or solids, which may be delivered or collected from the vessel for therapeutic or diagnostic purposes. Therapeutic agents may include biologically active substances, or substances capable of eliciting a biological response, including, but not limited to endogenous substances (growth factors or cytokines, including, but not limited to basic fibroblast growth factor, acidic fibroblast growth factor, vascular endothelial growth factor, angiogenic factors), viral vectors, DNA capable of expressing proteins, sustained release polymers, and unmodified or modified cells. Therapeutic agents may include angiogenic agents which induce the formation of new blood vessels. Therapeutic agents may also include anti-stenosis or anti-restenosis agents which are used to treat the narrowing of blood vessel walls. Therapeutic agents may include light-activated agents such as light-activated anti-stenosis or light-activated anti-restenosis agents that may be used to treat the narrowing of blood vessel walls.

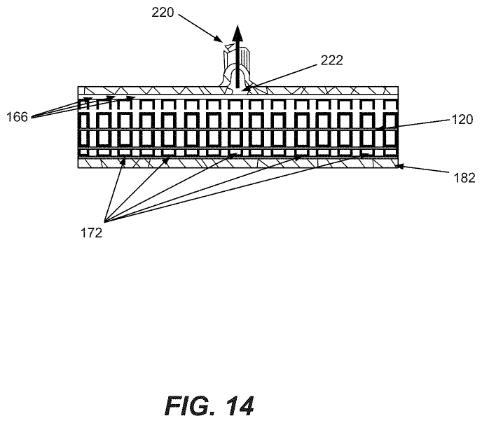

FIG. 14 is a side elevational view of an exemplary apparatus placed in a vessel of a subject, according to another exemplary embodiment of the present disclosure. The first distal balloon 120 may expand into contact with the vessel 182 and seal the confined volume 172 against the vessel wall 182, minimizing drug loss to smaller vessels, such as side branches or collaterals 220. In some embodiments, the longitudinal and circumferential surfaces (e.g. 168, 166 may contact the vessel wall 182 and seal the confined volume 172. When the entire vessel is filled with a drug, it will escape through any exit, side branch or collateral. The first distal balloon 120 moves the slitted apertures (e.g. 198) away from the vessel wall 182 and fills the confined volume 172. If the confined volume 172 is near an opening 222 (e.g. collateral 220), the small amount of drug that is transmitted through that confined volume 172 would be lost to the opening. If the confined volume 172 is not near an opening 222, the drug may be delivered to the vessel wall 182. The slitted apertures 198 may be distant from the vessel wall 182 inside the confined volume 172 and protected from potential environment debris. The drug flow from the slitted apertures 198 is not impeded by the slitted apertures 198 contacting the vessel wall 182.

Another embodiment of this disclosure includes an exemplary method of tissue restoration in a blood vessel of a subject. The method may include providing a catheter into the blood vessel. In some embodiments, the catheter may include the features of apparatus 100 described above. For example, the catheter may include a catheter shaft (e.g. catheter shaft 104) extending from a proximal end (e.g. proximal end 106) to a distal tip (e.g. distal tip 110). A first distal balloon (e.g. first distal balloon 120) may be positioned on a translucent distal segment (e.g. distal segment 130) of the catheter shaft proximal to the distal tip, the first distal balloon in fluid communication with a drug source via a first lumen (e.g. first distal balloon inflation lumen 150). The first distal balloon may include a translucent material, a first outer surface (e.g. first outer surface 124) positioned at a first radial distance (e.g. R1) from a center of the first distal balloon, a second outer surface (e.g. second outer surface 126) positioned at a second radial distance (e.g. R2) from the center of the first distal balloon, the second radial distance being larger than the first radial distance. The first distal balloon may have a patterned outer profile formed by the first outer surface and the second outer surface. The catheter may further include a second distal balloon (e.g. second distal balloon 122) positioned inside of and concentric with the first distal balloon, the second distal balloon in fluid communication with a second lumen (e.g. second distal balloon inflation lumen 154) separate from the first lumen. The catheter may further include a first light fiber (e.g. light fiber 140) and a second light fiber (e.g. light fiber 142) each positioned in the catheter shaft and extending through the translucent distal segment.

The method may further include partially expanding the second distal balloon, supplying the drug from the drug source to the first distal balloon, expanding the first distal balloon into contact with the blood vessel (e.g. blood vessel 182) in a treatment area; delivering the drug to the treatment area through at least one slitted aperture (e.g. slitted apertures 198, 200) in the first distal balloon; fully expanding the second distal balloon propping open the vessel, activating the first light fiber and second light fiber thereby providing light transmission through the distal segment, the first distal balloon, and the second distal balloon to activate the drug in the treatment area. The light transmission to the treatment area may activate the NVS, which may be activated by light. The expansion of the first distal balloon may shape the treatment area (e.g. vessel) as desired.

In some embodiments, the method may include engaging and separating plaque (e.g. plaque 180) along a wall of a vessel of a subject into smaller, less pressure resistant and isolated sections using the longitudinal and circumferential surfaces (e.g. 168, 166) of the patterned outer profile. The longitudinal and circumferential surfaces (e.g. 168, 166) may be interconnected and define at least one confined volume (e.g. volume or well 172) on the outer surface of the first distal balloon, the confined volume defined by a difference in radial distance (e.g. difference between R2 and R1) from the center of the first distal balloon between the first outer surface and the second outer surface. The method may further include creating isolated plaque sections that allow the drug to penetrate the vessel wall through gaps between each of the isolated plaque sections. The method may further include expanding the second distal balloon thereby expanding the first distal balloon as an outer surface of the second distal balloon contacts an inner surface of the first distal balloon.

Accordingly, the apparatus and methods described herein provide the delivery of NVS to a treatment area (e.g. a vessel) and provide restoration to that treatment area using the apparatus or according to the methods described above. The apparatus and method described above provide concurrently scoring the vessel, treating the vessel with one or more drugs (e.g. with Paclitaxel and NVS) with minimal loss to other vessels, scaffolding and casting the vessel, and light activation of the one or more drugs delivered to the treatment area. These advantages can be accomplished utilizing the apparatus and methods described herein.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware and software, but systems and methods consistent with the present disclosure can be implemented as hardware alone. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations and/or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps and/or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. An apparatus comprising
a catheter shaft extending from a proximal end to a distal tip;
a first distal balloon positioned on a translucent distal segment of the catheter shaft proximal to the distal tip, the first distal balloon in fluid communication with a drug source via a first lumen, the first distal balloon comprising:
a translucent material;
a first outer surface positioned at a first radial distance from a center of the first distal balloon;
a second outer surface positioned at a second radial distance from the center of the first distal balloon, the second radial distance being larger than the first radial distance;
a patterned outer profile of first distal balloon formed by the first outer surface and the second outer surface;
a second distal balloon positioned inside of and concentric with the first distal balloon, the second distal balloon in fluid communication with a second lumen separate from the first lumen; and
a first light fiber and a second light fiber each positioned in the catheter shaft and extending through the translucent distal segment;
wherein the drug source provides at least one drug to the first distal balloon via the first lumen.

2. The apparatus of claim 1, wherein patterned outer profile formed by the second outer surface includes longitudinal zones favoring less resistance than other areas of the first distal balloon, the longitudinal zones permitting the first distal balloon to selectively fold along these zones as the first distal balloon is compressed into a smaller shape.

3. The apparatus of claim 1, wherein the patterned outer profile includes longitudinal and circumferential surfaces that, with the first distal balloon expanded, engage and separate plaque along a wall of a vessel of a subject into smaller, less pressure resistant and isolated sections.

4. The apparatus of claim 3, wherein the longitudinal and circumferential surfaces are interconnected and define at least one confined volume on the outer surface of the first distal balloon, the confined volume defined by a difference in radial distance from the center of the first distal balloon between the first outer surface and the second outer surface.

5. The apparatus of claim 4, wherein expanding the first distal balloon seals the confined volume against the vessel wall, minimizing drug loss to smaller vessels, side branches or collaterals.

6. The apparatus of claim 4, wherein expanding the first distal balloon creates isolated plaque sections allowing the drug to penetrate a vessel wall through gaps between each of the isolated plaque sections.

7. The apparatus of claim 1, wherein the first outer surface includes slitted apertures that communicate the drug from the first distal balloon to a treatment area of a subject.

8. The apparatus of claim 1, wherein the second outer surface includes slitted apertures that communicate the drug from the first distal balloon to a treatment area of a subject.

9. The apparatus of claim 1, wherein the first distal balloon comprises a confined volume in fluid communication with the proximal end of the catheter shaft.

10. The apparatus of claim 9, wherein the confined volume is in fluid communication with at least one slitted aperture extending through from an interior side of the first distal balloon to an exterior side of the first distal balloon providing fluid communication from the interior side of the first distal balloon to the exterior side of the first distal balloon.

11. The apparatus of claim 10, wherein the at least one slitted aperture is in fluid communication with the drug source, the drug source supplying a drug into the confined volume in the first distal balloon and through the slitted aperture to the exterior side of the first distal balloon.

12. The apparatus of claim 11, wherein a volume pressure of the first distal balloon increases and inflates the first distal balloon, the increased volume pressure forces edges of the slitted aperture to open apart thereby reducing the balloon pressure.

13. The apparatus of claim 1, wherein the second outer surface of the first distal balloon contains a drug secured to the surface permitting the simultaneous delivery of two distinct and separate drugs.

14. The apparatus of claim 1, wherein expanding the second distal balloon consequently expands the first distal balloon as an outer surface of the second distal balloon contacts an inner surface of the first distal balloon.

15. The apparatus of claim 1 wherein the translucent material of the distal segment, the first distal balloon, and the second distal balloon is transparent.

16. The apparatus of claim 1 wherein the first light fiber and the second light fiber provide light activation through the distal segment, the first distal balloon, and the second distal balloon.

17. A method of tissue restoration in a blood vessel of a subject comprising:
providing a catheter into the blood vessel, the catheter comprising:
a catheter shaft extending from a proximal end to a distal tip;
a first distal balloon positioned on a translucent distal segment of the catheter shaft proximal to the distal tip, the first distal balloon in fluid communication with a drug source via a first lumen, the first distal balloon comprising:
a translucent material;
a first outer surface positioned at a first radial distance from a center of the first distal balloon;
a second outer surface positioned at a second radial distance from the center of the first distal balloon, the second radial distance being larger than the first radial distance;
a patterned outer profile of first distal balloon formed by the first outer surface and the second outer surface;
a second distal balloon positioned inside of and concentric with the first distal balloon, the second distal balloon in fluid communication with a second lumen separate from the first lumen; and
a first light fiber and a second light fiber each positioned in the catheter shaft and extending through the translucent distal segment;
supplying a drug from the drug source to the first distal balloon;
partially expanding the second distal balloon;
expanding the first distal balloon into contact with the blood vessel in a treatment area;
delivering the drug to the treatment area through at least one slitted aperture in the first distal balloon;
fully expanding the second distal balloon;
activating the first light fiber and the second light fiber thereby providing light transmission through the distal segment, the first distal balloon, and the second distal balloon to activate the drug in the treatment area.

18. The method of claim 17, further comprising:
engaging and separating plaque along a wall of a vessel of a subject into smaller, less pressure resistant and isolated sections using the longitudinal and circumferential surfaces of the patterned outer profile.

19. The method of claim 17, wherein the longitudinal and circumferential surfaces are interconnected and define at least one confined volume on the outer surface of the first distal balloon, the confined volume defined by a difference in radial distance from the center of the first distal balloon between the first outer surface and the second outer surface.

20. The method of claim 17, further comprising:
creating isolated plaque sections that allow the drug to penetrate a vessel wall through gaps between each of the isolated plaque sections.

21. The method of claim 17, further comprising:
expanding the second distal balloon thereby expanding the first distal balloon as an outer surface of the second distal balloon contacts an inner surface of the first distal balloon.

22. An apparatus comprising
a catheter shaft extending from a proximal end to a distal tip;
a first distal balloon positioned on a translucent distal segment of the catheter shaft proximal to the distal tip, the first distal balloon in fluid communication with a drug source via a first lumen, the first distal balloon comprising:
a translucent material;
a first outer surface positioned at a first radial distance from a center of the first distal balloon;
a second outer surface positioned at a second radial distance from the center of the first distal balloon, the second radial distance being larger than the first radial distance;
a patterned outer profile of first distal balloon formed by the first outer surface and the second outer surface, the patterned outer profile including interconnected longitudinal and circumferential surfaces that define at least one confined volume on the outer surface of the first distal balloon, the confined volume defined by a difference in radial distance from the center of the first distal balloon between the first outer surface and the second outer surface;
a first light fiber and a second light fiber each positioned in the catheter shaft and extending through the translucent distal segment;
wherein the drug source provides at least one drug to the first distal balloon via the first lumen
the interconnected longitudinal and circumferential surfaces, with the first distal balloon expanded, engage and separate plaque along a wall of a vessel of a subject into smaller, less pressure resistant and isolated sections.
